(19) United States
(12) Patent Application Publication
(10) Pub. No.: US 2006/0100606 A1
(43) Pub. Date: May 11, 2006

(54) Method and device for liposuction

(76) Inventor: John D. Dobak III, La Jolla, CA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 11/255,474

(22) Filed: Oct. 21, 2005

Related U.S. Application Data
(60) Provisional application No. 60/626,579, filed on Nov. 10, 2004.

(51) Int. Cl. A61M 1/00

(52) U.S. Cl. 604/542

(57) ABSTRACT

Systems and methods for liposuction are disclosed that use a lipid-emulsifying fluid to produce fluid jet disruption and Venturi pumping of fat tissue. The device includes a flexible or rigid probe with an inlet lumen and an outlet lumen. The inlet lumen is in fluid communication with a pump capable of producing high pressures, e.g., 250-3500 psi. The pump is in fluid communication with a volume of a liquid such as sterile saline that may or may not contain a fat emulsifier. At the end of the inlet lumen is a conical constriction or a port that accelerates the emulsification fluid to high velocities. The high velocity jet stream creates a low pressure area that draws the fat tissue into proximity with the jet stream. The jet stream directly and through turbulent vortices breaks up the fat cells. The lipid of the fat cells is subsequently emulsified by the emulsification fluid.

### CROSS-REFERENCE TO RELATED APPLICATIONS

This application claims the benefit of priority of U.S. Provisional Patent Application Ser. No. 60/626,579, filed Nov. 10, 2004, entitled “Method and Device for Liposuction”.

### BACKGROUND

Liposuction is performed to remove subcutaneous fat from the body as a cosmetic procedure to improve appearance. Traditional liposuction is performed using a suction cannula that is moved in a continuous fashion in the subcutaneous tissue to disrupt fat cells and evacuate the debris. Cells are disrupted mechanically. In tumescent techniques, saline mixed with a vasoconstricting drug such as epinephrine is injected into the fat tissue prior to suction. Other techniques to facilitate liposuction include ultrasonic disruption of cells, probe vibration, moving scalpel blades, and laser illumination. All of these techniques suffer similar problems, which include excessive blood loss, skin dimpling and irregularities, perforation, and skin burns. In addition, current techniques can damage blood vessels and nerves.

These problems limit the use of liposuction in delicate areas such as the periorbital fat region, where sagging and bulging tissue can form. Traditional liposuction probes, which may be, e.g., 4-8 mm in diameter, are too large and the mechanical forces required to disrupt the fat tissue can damage surrounding structures. For similar reasons, liposuction of the face in general is even more precarious using traditional instrumentation.

The above-mentioned problems with current liposuction systems also prevent liposuction from being performed in the peritoneal cavity. Significant peritoneal or visceral fat accumulation is associated with severe medical conditions such as diabetes, hypertension, and hypercholesterolemia. Much of the fat in the peritoneal cavity is found on the mesenteries and omentum. Omentectomy, or the removal of the omentum, is associated with improved outcomes after gastric banding. Liposuction that can safely and effectively remove fat tissue from the abdomen may improve medical conditions associated with its accumulation.

Saline jet systems are used to remove thrombus from the arteries of the body and to remove spinal disc nuclear material. Saline jets have been used to dissect fat tissue as a probe is pushed through the fat. This jet spray is directed away from the probe or cannula.

### SUMMARY OF THE INVENTION

Systems and methods for liposuction are disclosed that use a lipid-emulsifying fluid to produce fluid jet disruption and Venturi pumping of fat tissue. The device includes a flexible or rigid probe with an inlet lumen and outlet lumen. The inlet lumen is in fluid communication with a pump capable of producing high pressures, e.g., 250-3500 psi. The pump is in fluid communication with a volume of a liquid such as sterile saline that may or may not contain a fat emulsifier such as a phospholipid or bile salt or a mixture of the two. The outlet lumen is in fluid communication with a collection reservoir. At the end of the inlet lumen is a conical constriction, exhaust outlet, or port that accelerates the emulsification fluid to high velocities. The high velocity jet stream creates a low pressure area that draws the fat tissue into proximity with the jet stream. The jet stream directly and through turbulent vortices breaks up the fat cells. The lipid of the fat cells is subsequently emulsified by the emulsification fluid. The emulsified fat tissue is then evacuated from the body due to the action of the high velocity streaming of the emulsification fluid into the outlet lumen. A vacuum pump or suction syringe, which is optional, can be used to facilitate this evacuation.

Advantages of certain systems may include one or more of the following. Certain systems may safely improve the precision of liposuction. Certain systems may facilitate microliposuction in areas of the face such as the periorbital region with probes less than 2.0 mm in diameter. Certain systems may help preserve blood vessels and nerves. Certain systems may safely allow intraperitoneal liposuction.

Additional advantages will become apparent from the description that follows, including the drawings and claims.

### BRIEF DESCRIPTION OF THE DRAWINGS

FIG. 1 illustrates a system according to an embodiment of the invention.

FIG. 2 illustrates a more detailed view of the cannula of the system of FIG. 1.

FIG. 3 illustrates a system that may be employed using multiple jet streams.

### DETAILED DESCRIPTION

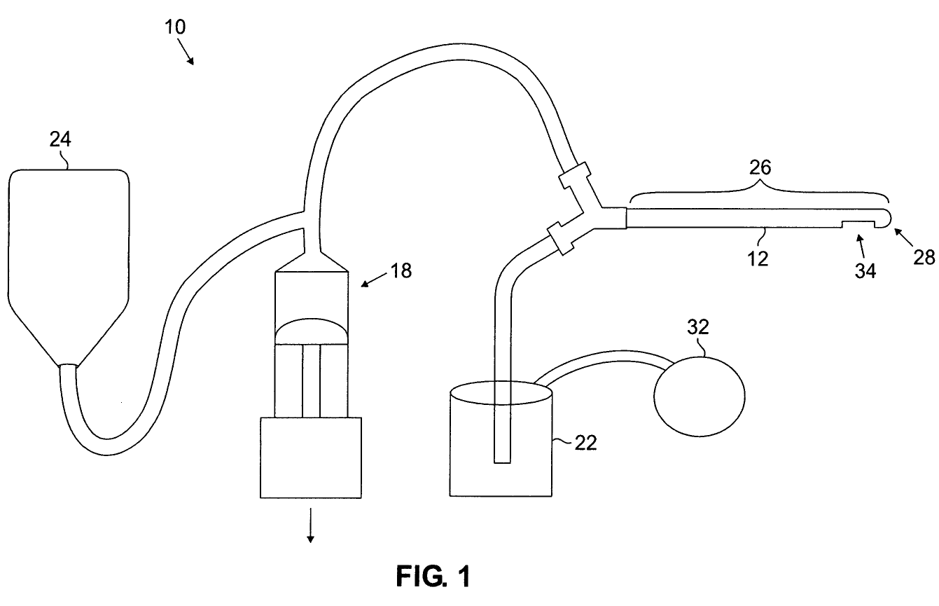

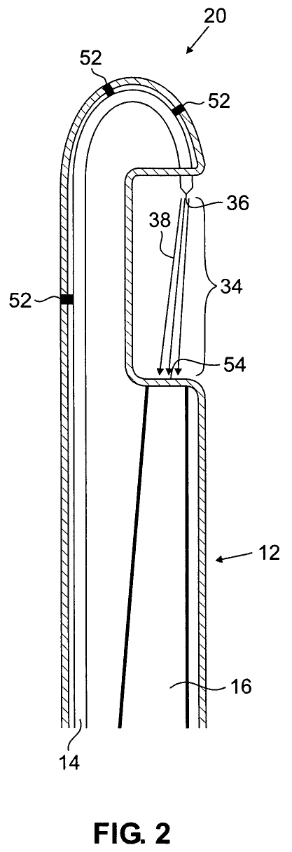

Referring to FIGS. 1 and 2, an integrated system 10 includes a cannula 12 with a high pressure inlet lumen 14 and a lower pressure outlet lumen 16, a high pressure pump or source of compressed gas 18, an evacuation container or reservoir 22, and a source of liquid such as a fat emulsifying solution 24. An optional vacuum pump 32 may also be employed.

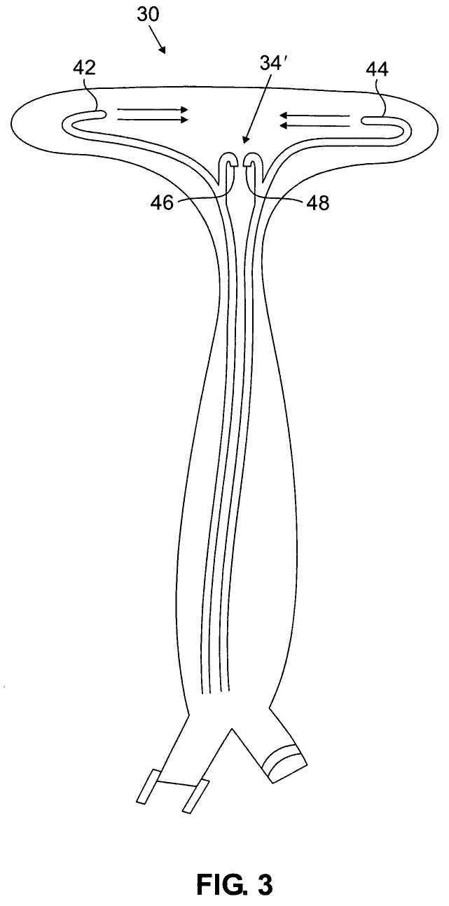

The cannula 12 typically has a body 26 of variable length depending on the procedure and a blunt tip 28. The cannula diameter may range from 1.5-8.0 mm and may be shaped to facilitate liposuction in a given area. For example, facial liposuction may be conducted with a cylindrical probe 20 as shown in FIG. 2. Referring to FIG. 3, intraperitoneal liposuction may be conducted with a curved cylindrical probe or a probe 30 that flattens out in the horizontal plane similar to a vacuum cleaner. An exhaust outlet 36 is structured and configured to create a jet spray 38, as explained in greater detail below. Just proximal to the rounded or blunt tip, and adjacent the exhaust outlet 36, is a tissue entrainment orifice or port 34 or 34′ into which fat is suctioned and emulsified. For cylindrical cannula, the port is preferably located on one side of the cannula, as shown in FIG. 2, such that the cannula can be rotated to one side thereby shielding other tissue from the suction/emulsification action.

As noted above in connection with FIG. 3, the cannula 20 may have multiple suction/emulsification ports. In Ports 42 and 44 include inlet lumens and exhaust outlets which create horizontal jet sprays to facilitate disruption of fat. The ports need not be perfectly horizontal—depending on the tissue to be subject to the procedure, it may be desired to angle the ports. Ports 46 and 48 form return lumen jet sprays, as the same facilitate removal and suction of emulsified fat away from the entrainment orifice or port.

In addition, the port may exist at the end of a cannula. Numerous other geometries may also be envisioned.

The cannula may be rigid or flexible depending on the location of use. In general rigid probes could be used subcutaneously and flexible probes could be used intraperitoneally.

One or more high pressure lumens, each with an inlet and an outlet resides within the cannula extending from a manifold that allows connections of the various lumens to either the high pressure pump or the high pressure source, e.g., a source of compressed gas, or the evacuation reservoir. The high pressure lumens may be made from drawn stainless steel tubing and have sufficient wall strength to support pressures from 350-3500 psi. The diameter of the higher pressure tubing may be large enough to allow flow rates of 2-50 ml/min at a given or desired inlet pressure. The exhaust outlet 36 of the high pressure lumen is a small hole or conical-shaped taper in the tubing. The shape and size of the outlet are suitable for creating a high velocity jet and its attendant pressure changes that allow tissue to be suctioned into the stream. The pressure in the lumen 14 just prior to the outlet is preferably 100 to 350 psi to greater than 1,000 psi. The outlet lumen runs parallel or coaxial, in which case it may be the outer lumen, with the inlet lumen in the body of the cannula. The outlet lumen 16 has an inlet 54 and is of sufficient size to allow the removal of the emulsified fat tissue. In one embodiment, at the tip 28 of the cannula, the high pressure or inlet lumen 14 turns back on itself such that the jet is directed toward the outlet lumen 16. The inlet lumen's outlet may reside inside, near, or at a distance from the outlet lumen. Preferably the exhaust outlet of the inlet lumen resides close enough to the outlet lumen to allow the creation of suction which will draw fat tissue near or into the cannula tip, but far enough away to allow the jet stream to disrupt and emulsify the tissue. The distance to accomplish this may be from approximately 0.5 mm to 5 mm. It may also be possible to have several high pressure inlet lumens, jet-spray-creating outlets, and jet sprays, some of which are used to create the suction effect and others which disrupt and emulsify the tissue.

The inlet lumen 14 may also have side ports 52 proximal to the distal jet port or exhaust outlet 36. These ports allow a low-velocity and low-pressure injection of fluid into the fat tissue to assist the emulsification process prior to evacuation. Thus, as the cannula is moved across the tissue, the fat emulsification may occur just prior to suction, disruption, and evacuation by the fluid jet.

The inlet lumen 14 of the cannula 20 is in fluid communication with a pump 18. This pump is preferably a piston-type pump that is disposable and capable of creating pressures of 350 psi or greater. The pump 18 is in fluid communication with a source 24 of physiologic saline or a fat-emulsifying or fatsoluble solution. The shear and turbulent forces created by a saline jet stream may be sufficient to cause the fat tissue removal. One or more lipid emulsifiers may be added to improve the tissue removal. In general, lipid emulsifiers typically have an ionic region that is soluble in water and a hydrophobic region that is soluble in fat. Representative lipid emulsifiers are phospholipids, such as phosphatidyl choline, and bile salts, such as sodium deoxycholate. With phospholipids, the ionic region is a phosphate molecule and the hydrophobic region a fatty acid. Alternatively, lipid-soluble fluids such as hydrocarbons, perflourocarbons, or fluorocarbons may be used individually or in mixtures. Any fluid used should have appropriate biocompatibility.

One representative solution is a mixture in water of phosphatidyl choline (1-5% weight/volume) and sodium deoxycholate (1-5% weight/volume). Benzyl alcohol, an antimicrobial, may be added to the mixture, e.g., at a 0.9% weight/volume. This fluid can be sterilized and is biocompatible, and is known to be safe as the same has been used in medical procedures, such as subcutaneous injection for lipolysis.

While the invention has been described in terms of specific embodiments, it will be clear to one of ordinary skill in the art, given this teaching, that variations of the above are also within the scope of the invention. The scope of the invention is to be interpreted solely by the claims appended hereto.

What is claimed is:

1. A device for removing fat from the body:
A cannula having an inlet lumen and an outlet lumen, the inlet lumen having an exhaust port at a distal end thereof;
A source of high pressure liquid, said source coupled to said inlet lumen;
Wherein the exhaust outlet of the inlet lumen is structured and configured to create a high velocity jet stream of said liquid;
wherein said jet stream creates a region of low pressure so as to bring fat in proximity of the jet stream; and
wherein said jet stream is sufficient to disrupt tissue for removal.

2. The device of claim 1, wherein said device is used to remove intraperitoneal fat tissue, periorbital fat tissue, facial fat tissue.

3. The device of claim 1, wherein said liquid contains up to 5% of phosphatidyl choline per 100 ml of water.

4. The device of claim 1, wherein said liquid contains up to 5% of sodium deoxycholate per 100 ml of water.

5. The device of claim 1, wherein said liquid contains a mixture of 5% phosphatidyl choline and 4.75% sodium deoxycholate per 100 ml of water.

6. The device of claim 1, wherein said liquid is physiologic saline.

7. A device for removing fat from the body:
A cannula having an inlet lumen and an outlet lumen, the inlet lumen having an exhaust outlet at a distal end thereof, the distance between the exhaust outlet and the inlet of the outlet lumen close enough that sufficient suction is created to draw fat tissue near or into the cannula tip, but far enough away to allow the jet stream to disrupt and emulsify the tissue;
a source of high pressure liquid, said source coupled to said inlet lumen;
wherein the exhaust outlet of the inlet lumen is structured and configured to create a high velocity jet stream of said liquid;
wherein said jet stream creates a region of low pressure so as to bring fat in proximity of the jet stream; and
wherein said jet stream is sufficient to disrupt tissue for removal.

8. The device of claim 7, wherein the distance is from approximately 0.5 mm to 5 mm.

9. A method for removing fat from the body:
Inserting a cannula having an inlet lumen and an outlet lumen, the inlet lumen having an exhaust outlet at a distal end thereof, and disposing the exhaust outlet adjacent a location to be treated;
Pressurizing a source of high pressure liquid, said source coupled to said inlet lumen;
Creating a high-velocity jet stream of said liquid out of the exhaust outlet of the inlet lumen;
wherein said jet stream creates a region of low pressure so as to bring fat in proximity of the jet stream; and
wherein said jet stream is sufficient to disrupt tissue for removal.

10. The method of claim 9, wherein said location to be treated includes intraperitoneal fat tissue, periorbital fat tissue, or facial fat tissue.

11. The method of claim 9, wherein said liquid contains up to 5% of phosphatidyl choline per 100 ml of water.

12. The method of claim 9, wherein said liquid contains up to 5% of sodium deoxycholate per 100 ml of water.

13. The method of claim 9, wherein said liquid contains a mixture of 5% phosphatidyl choline and 4.75% sodium deoxycholate per 100 ml of water.

14. The method of claim 9, wherein said liquid is physiologic saline.
